(19) United States
(12) Patent Application Publication
(10) Pub. No.: US 2025/0169769 A1
(43) Pub. Date: May 29, 2025

(54) COMPRESSION EVENT DETECTION FOR CONTINUOUS GLUCOSE MONITORS

(71) Applicant: Dexcom, Inc., San Diego, CA (US)

(72) Inventors: Zachary Felix WOOD, San Diego, CA (US); Rui MA, San Diego, CA (US); Harleen Sarah Kaur SINGH, San Diego, CA (US); Anna C. HARLEY-TROCHIMCZYK, San Diego, CA (US); Qi AN, San Diego, CA (US)

(21) Appl. No.: 18/960,126

(22) Filed: Nov. 26, 2024

Related U.S. Application Data
(60) Provisional application No. 63/603,070, filed on Nov. 27, 2023.

(51) Int. Cl. A61B 5/00; A61B 5/145

(52) U.S. Cl. CPC A61B 5/7296; A61B 5/14532; A61B 5/14546; A61B 5/7246; A61B 5/7264; A61B 2562/0219

(57) ABSTRACT

A continuous analyte monitoring system includes first and second analyte sensors configured to sense analytes such as lactate and glucose in the tissue of a user. A controller Is coupled to the analyte sensors and configured evaluate first samples of outputs of the first analyte sensor and second samples of outputs of the second analyte sensor with respect to one another to determine whether the first samples and the second samples indicate compression of the tissue. If the first samples and the second samples indicate compression of the tissue, compensate for the compression of the tissue with respect to the first samples. The controller may evaluate the machine learning models using a machine learning model or a filter.

### CROSS-REFERENCE TO RELATED APPLICATIONS

This application claims priority to and benefit of U.S. Provisional Patent Application No. 63/603,070, filed Nov. 27, 2023, which is assigned to the assignee hereof and hereby expressly incorporated herein in its entirety as if fully set forth below and for all applicable purposes.

### BACKGROUND

Diabetes mellitus is a metabolic condition relating to the production or use of insulin by the body. Insulin is a hormone that allows the body to use glucose for energy, or store glucose as fat.

When a person eats a meal that contains carbohydrates, the digestive system absorbs nutrients, ultimately depositing glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), skin ulcers, and kidney damage. Hyperglycemia can also lead to acute problems, such as diabetic ketoacidosis-a state in which the body becomes excessively acidic due to the production of excess ketones, or body acids. The state of having lower than normal blood glucose levels is called “hypoglycemia.” Severe hypoglycemia can lead to damage of the heart muscle, neurocognitive dysfunction, and in certain cases, acute crises that can result in seizures or even death.

A patient living with diabetes can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Patients with diabetes can benefit from real-time diabetes management guidance, as determined based on a physiological state of the patient, in order to stay within a target glucose range and avoid physical complications. In certain cases, the physiological state of the patient is determined using diagnostics systems that measure glucose levels, which inform the identification and/or prediction of adverse glycemic events, such as hyperglycemia and hypoglycemia, and the type of guidance provided to the patient.

For example, such diagnostics systems may utilize a continuous glucose monitor (CGM) to measure a patient's glucose levels over time. The measured glucose levels may then be processed by the diagnostics system to identify and/or predict adverse glycemic events, and/or to provide guidance to the patient for treatment and or actions to abate or prevent the occurrence of such adverse glycemic events. For example, trends, statistics, or other metrics may be derived from the glucose levels and used to identify and/or predict adverse glycemic events. Or, in certain cases, the glucose levels themselves may be used to identify and/or predict adverse glycemic events.

A CGM includes a sensor mounted to the body of the patient, such as to the arm or torso. The sensor includes electrodes coated in enzymes that are in contact with the blood and/or interstitial fluid of the patient, each enzyme reacts with an analyte to be sensed, such as glucose, lactate, or others. When an analyte reacts with the enzyme on an electrode, a detectable current is induced with the magnitude of the current corresponding to the concentration of the analyte.

The detected current from a given electrode may be influenced by factors other than the concentration of the analyte corresponding to the enzyme coating the electrode. The sensor may also unintentionally detach from the patient. The electrodes or circuits connected to them may be damaged. Absent a failure or detachment of the sensor itself, the detected current may also be affected by compression of the sensor itself due to the patient sitting or lying on the sensor or some other source of pressure. Compression of the sensor may cause the output of the sensor to inaccurately indicate sensor failure.

Accordingly, there is a need in the art for improved systems and methods for accurately detecting failure of a CGM sensor.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates aspects of an example disease management system used in connection with implementing embodiments of the present disclosure.

FIG. 2 is a diagram conceptually illustrating an example continuous analyte monitoring system including example continuous analyte sensors with sensor electronics, in accordance with certain aspects of the present disclosure.

FIG. 3 is a block diagram illustrating electrodes of a continuous analyte monitoring system, according to certain embodiments disclosed herein.

FIG. 4 is a block diagram that illustrates electronics associated with the continuous analyte monitoring system of FIG. 2, according to certain embodiments disclosed herein.

FIG. 5 illustrates plots of the current output by glucose and lactate analyte sensors during a compression event, according to certain embodiments disclosed herein.

FIG. 6 illustrates plots of the current output by glucose and lactate analyte sensors in response to an acute compression event, according to certain embodiments disclosed herein.

FIG. 7 is a diagram illustrating the flow of data with respect to a sensor failure detection module, according to certain embodiments disclosed herein.

FIG. 8 illustrates the use of a machine learning model to identify compression events, according to certain embodiments disclosed herein.

FIG. 9 is a process flow diagram of a method for determining whether to perform processing to compensate for compression events, according to certain embodiments disclosed herein.

FIG. 10 illustrates a method for performing compression filtering, according to certain embodiments disclosed herein.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

As described above, compression of an analyte sensor may cause the output of an analyte sensor to inaccurately measure an analyte, such as glucose. Therefore, a technical problem or deficiency in existing continuous analyte monitoring systems is that, during compression, such systems may generate inaccurate analyte measurements.

Accordingly, systems and methods according to the present principles provide a technical solution to the technical problem described above by detecting and compensating for compression events for a continuous analyte monitoring system. More particularly, embodiments provided herein include improved systems and methods for detecting compression events using multiple analyte sensors, such as for sensing glucose and lactate. Such systems and methods help avoid disturbing the patient with false alarms while still promoting patient safety. Accordingly, the present disclosure provides a technical solution to the problems described above by providing an approach for accurately detecting compression events.

Specifically, a continuous analyte monitoring system is described that evaluates both lactate and glucose samples. For example, in response to detecting a force exceeding a threshold, expected values for lactate and glucose samples may be compared to the actual lactate and glucose samples. The expected values may be calculated using a filter, which may use a function of past values for the glucose and lactate samples and possibly other inputs such as force, exercise, diet, and drug data. If differences between the actual lactate and glucose samples have an inverse correlation with respect to the expected values for the lactate and glucose sample, a compression event may be deemed to have occurred and the glucose samples may be adjusted. If an inverse correlation is not found, an acute compression event may be deemed to have occurred and the glucose samples may be blanked.

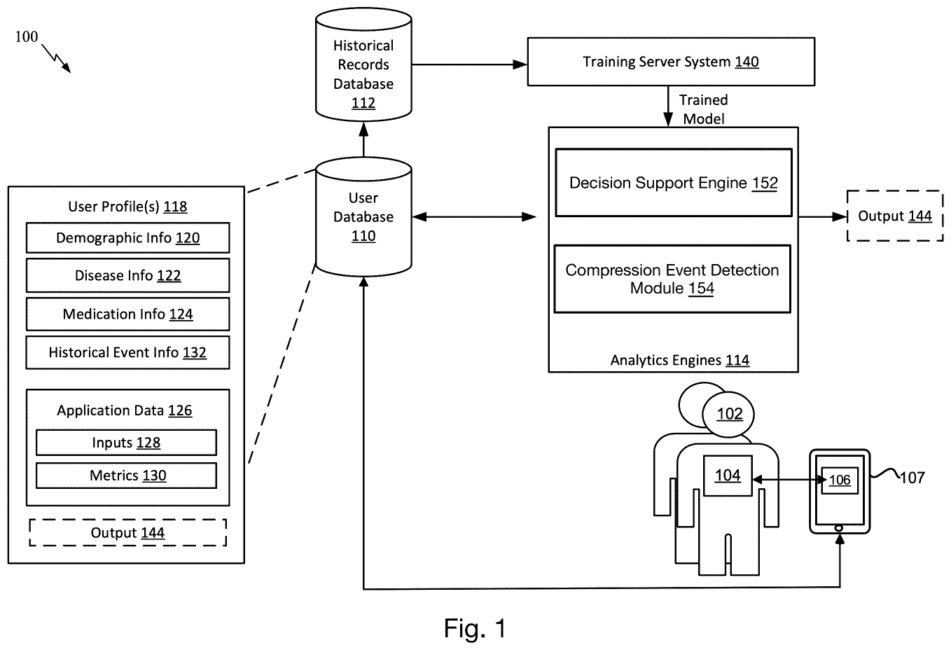

FIG. 1 illustrates an example disease management system 100 for assisting users 102 (individually referred to herein as a user and collectively referred to herein as users) with decision support for managing a disease, e.g., diabetes, kidney disease, liver disease, or other types of diseases. The system 100 utilizes a continuous analyte monitoring system 104 that continuously measures one or more of a plurality of analytes, such as glucose, lactate, potassium, creatinine, ketone, alcohol, etc. A user may be a patient or, in some cases, the patient's caregiver. In certain embodiments, the system 100 includes the continuous analyte monitoring system 104, a display device 107 that executes an application 106, analytics engines 114, a user database 110, a historical records database 112, and a training server system 140, each of which is described in more detail below.

The analytes that may be measured and analyzed by the devices and methods described herein include glucose, lactate, ketones, potassium, and in some examples, other analytes listed above. However, other analytes, which are not listed above, may also be considered.

An analyte may be a substance or chemical constituent in a biological fluid (for example, blood, interstitial fluid, cerebral spinal fluid, lymph fluid, sweat, or urine) that can be analyzed. Analytes can include naturally occurring substances, artificial substances, metabolites, or reaction products. Analytes for measurement by the devices and methods may include, but may not be limited to, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-3 hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycerol; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; potassium, quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

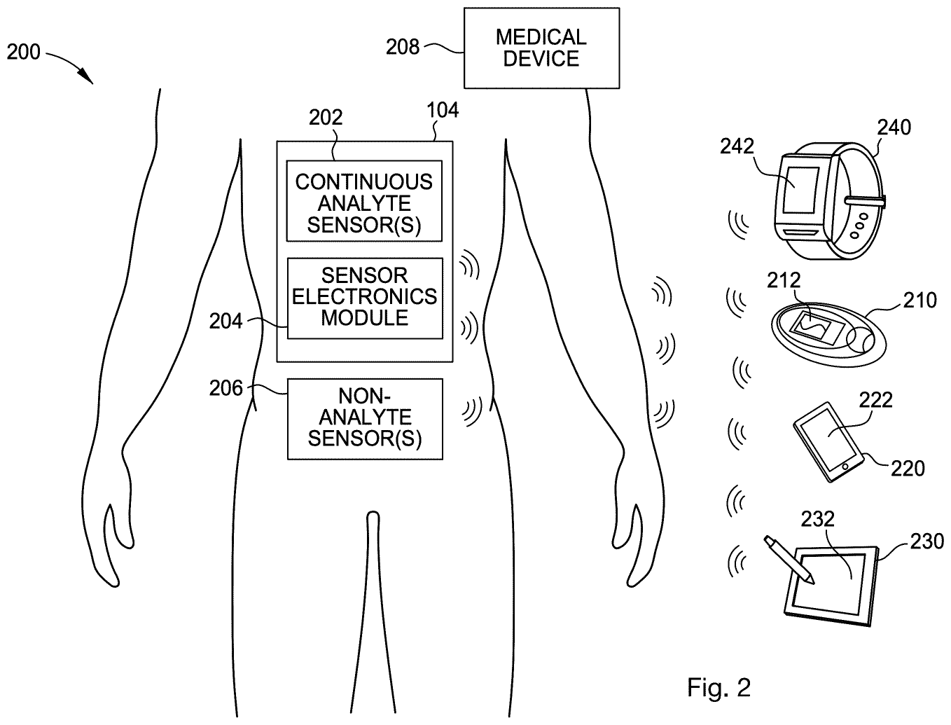

The continuous analyte monitoring system 104 may continuously measure one or more analytes and transmit the analyte measurements to the display device 107 for use by the application 106. In some embodiments, the continuous analyte monitoring system 104 transmits the analyte measurements to the display device 107 through a wireless connection (e.g., Bluetooth connection). The display device 107 may be a smart phone, a laptop computer, a smart watch, a fitness tracker, a tablet, or any other computing device capable of executing the application 106. The continuous analyte monitoring system 104 may be described in more detail with respect to FIG. 2.

The application 106 may be a mobile health application that receives and analyzes analyte measurements from the analyte monitoring system 104. For example, the application 106 stores information about a user, including the user's analyte measurements, in a user profile 118 of the user for processing and analysis, as well as for use by the decision support engine 152 to provide decision support recommendations or guidance to the user.

The analytics engines 114 include a decision support engine 152 providing disease management decision support recommendations to the user, e.g., via the application 106. The decision support engine 152 may provide such recommendations based on analyte measurements for one or more analytes received from the continuous analyte monitoring system 104, data obtained from one or more non-analyte sensors or devices, or information included in the user profile 118. In certain embodiments, the analytics engines 114 execute entirely on one or more computing devices in a private or a public cloud. In such embodiments, the application 106 communicates with the analytics engines 114 over a network (e.g., Internet). In some other embodiments, the analytics engines 114 execute partially on one or more local devices, such as the display device 107 or a processor of the continuous analyte monitoring system 104, and partially on one or more computing devices in a private or a public cloud. In some other embodiments, the analytics engines 114 execute entirely on one or more local devices, such as the display device 107 or a processor of the continuous analyte monitoring system 104.

The analytics engines 114 may include a compression event detection module 154. The compression event detection module 154 analyzes signals from the continuous analyte monitoring system 104 and determines whether the signals indicate a compression event. The compression event detection module 154 further implements methods described herein for distinguishing between compensatable compression events and acute compression events. During a compression event, compression of the tissue around the continuous analyte monitoring system 104 may cause the signals output therefrom to temporarily be very low or otherwise outside the range of values expected during normal operation. The compression event detection module 154 is therefore configured to identify such compression events and, in response, adjust and/or blank data recorded during compression events that is passed on to the decision support engine 152 or other consumer of the output of the continuous analyte monitoring system 104.

The user profile 118 may include information collected about the user from the application 106. For example, the application 106 provides a set of inputs 128, including the analyte measurements for the one or more analytes received from the continuous analyte monitoring system 104 that are stored in the user profile 118. In certain embodiments, inputs 128 provided by the application 106 include other data in addition to the analyte measurements. For example, the application 106 may obtain additional inputs 128 through manual user input, one or more other non-analyte sensors or devices (e.g., temperature sensors, etc.), other applications executing on the display device 107, etc. Non-analyte sensors and devices include one or more of, but are not limited to, an insulin pump, respiratory sensor, sensors or devices provided by the display device 107 (e.g., accelerometer, gyrometer, camera, global positioning system (GPS), heart rate monitor, etc.) or other user accessories (e.g., a smart watch), or any other sensors or devices that provide relevant information about the user. The inputs 128 of the user profile 118 provided by the application 106 may, for example, include continuous analyte sensor data, non-analyte sensor data, time, food consumption, physical activity, sleep information, user statistics, medication, etc.

The user profile 118 further includes demographic information 120, disease information 122, or medication information 124. Such information may be provided through user input or obtained from certain data stores (e.g., electronic medical records, etc.). The demographic information 120 may include one or more of the user's age, BMI (body mass index), ethnicity, gender, etc. The disease information 122 may include information about one or more diseases of a user, including relevant information pertaining to the user's condition of diabetes or other conditions (e.g., liver disease, kidney disease, etc.). The disease information 122 may also include the length of time since diagnosis, the level of disease control, level of compliance with disease management therapy, other types of diagnoses (e.g., heart disease, obesity), etc. The disease information 122 may include other measures of health (e.g., heart rate, stress, sleep, etc.) or fitness (e.g., cardiovascular endurance, muscular strength or power, muscular endurance, and other measures of fitness), or the like.

The medication information 124 may include information about the amount and type of a medication taken by a user. For example, the medication information 124 may include information about the consumption of one or more drugs for management of the user's condition of diabetes, such as insulin (e.g., short-acting insulin, rapid-acting insulin (insulin aspart, insulin gluilisine, insulin lispro), intermediate-acting insulin (insulin isophane), long-acting insulin degludec, indulin detemir, insulin glargine, insulin), combination insulins), amylinomimetic drugs, alpha-glucosidase inhibitors (e.g., acarbose, miglitol), biguanides (e.g., metformin-alogliptin, metformin-canagliflozin, metformin-dapagliflozin, metformin-empagliflozin, metformin-glipizide, metformin-glyburide, metformin-linagliptin, metformin-pioglitazone, metformin-repaglinide, metformin-rosiglitazone, metformin-saxagliptin, metformin-sitagliptin), dopamine agonists (e.g., bromocriptine), dipeptidyl peptidase-4 (DPP-4) inhibitors (e.g., alogliptin, alogliptin-pioglitazone, linagliptin, linagliptin-empagliflozin, saxagliptin, sitagliptin, simvastatin), glucagon-like peptide-1 receptor agonists (GLP-1 receptor agonists) (e.g., albiglutide, dulaglutide, exenatide, liraglutide, semaglutide), meglitinides (e.g., nateglinide, repaglinide), sodium-glucose transporter (SGLT) 2 inhibitors (e.g., dapagliflozin, canagliflozin, empagliflozin, ertugliflozin), sulfonylureas (e.g., glimepiride, glimepiride-pioglitazone, glimepiride-rosiglitazone, gliclazide, glipizide, glyburide, chlorpropamide, tolazamide, tolbutamide), thiazolidinediones (e.g., rosiglitazone, pioglitazone), and other drugs. The medication information 124 may include information about the consumption of one or more drugs for management or treatment of other diseases or conditions of the user, including drugs for cholesterol, high blood pressure, heart disease, etc., in addition to supplements to promote general health and metabolism, such as vitamins.

Data stored in the user profile 118 may maintain time series data collected for the user (e.g., the patient) over a period of time that the user utilizes the continuous analyte monitoring system 104 and the application 106. For example, analyte data for a user who has used the continuous analyte monitoring system 104 and the application 106 for a period of five years to manage their condition may have time series analyte data for the user maintained in the user profile 118 over the five-year period.

Further, data stored in the user profile 118 may provide time series data collected over the lifetime of the user. For example, the data may include information for the user that indicates physiological states of the user, glucose levels of the user, lactate levels of the user, ketone levels of user, states/conditions of one or more organs of the user, habits of the user (e.g., alcohol consumption, activity levels, food consumption, etc.), medications prescribed, etc., throughout the lifetime of the user.

The user profile 118 may be dynamic because at least part of the information that is stored in the user profile 118 may be revised or updated over time or new information may be added to user profile 118 by the analytics engines 114 or the application 106. Accordingly, the information in the user profile 118 stored in the user database 110 provides an up-to-date repository of information for the user.

The user database 110 may include a storage server that operates, for example, in a public or private cloud. The user database 110 may be implemented as any type of datastore, such as relational databases, non-relational databases, key-value datastores, file systems including hierarchical file systems, and the like. In some exemplary implementations, the user database 110 is distributed. For example, the user database 110 may include persistent storage devices, which are distributed. Furthermore, the user database 110 may be replicated so that the storage devices are geographically dispersed.

The user database 110 includes the user profiles 118 for multiple users, including users who similarly interact or have interacted in the past with the application 106 on their own devices. The user profiles stored in the user database 110 are accessible not only to the application 106, but to the analytics engines 114, as well. The user profiles in the user database 110 may be accessible to the application 106 and the analytics engines 114 over one or more networks (not shown). As described above, the analytics engines 114 can fetch inputs 128 from a user's profile 118 stored in the user database 110 and compute one or more metrics 130, which can then be stored as application data 126 in the user's profile 118.

The user profiles 118 stored in the user database 110 may also be stored in the historical records database 112. The user profiles 118 stored in the historical records database 112 may provide a repository of up-to-date information and historical information for each user of the application 106. Thus, the historical records database 112 essentially provides all data related to each user of the application 106, where data is stored using timestamps. The timestamp for any piece of information stored in the historical records database 112 may identify, for example, when the piece of information was obtained or updated.

Further, the historical records database 112 may include data for one or more patients who are not users of the continuous analyte monitoring system 104 or the application 106. For example, the historical records database 112 may include information (e.g., user profiles) for one or more patients analyzed by, for example, a healthcare physician (or other known method), who may or may not be diagnosed with diabetes. Data stored in the historical records database 112 may be referred to herein as population data.

Although depicted as separate databases for conceptual clarity, the user database 110 and the historical records database 112 may operate as a single database. The single database may be a storage server that operates in a public or private cloud.

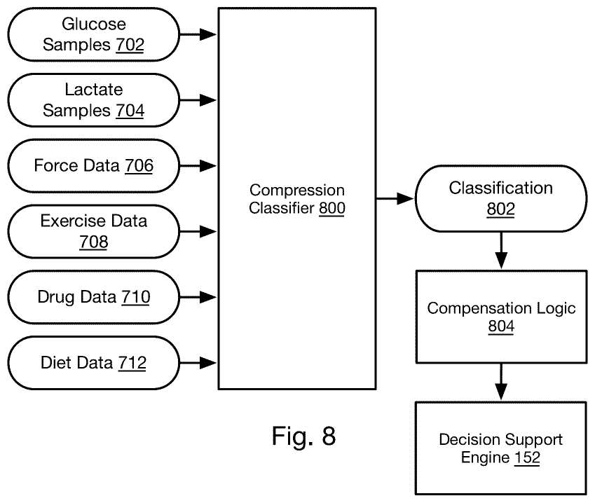

The training server system 140 may train the one or more machine learning models using training data, which may include data (e.g., from user profiles) for one or more patients (e.g., users or non-users of the continuous analyte monitoring system 104 or the application 106, e.g., diabetic patients). The training data may be stored in the historical records database 112 and may be accessible to the training server system 140 over one or more networks (not shown) for training the machine learning models. In particular, the training server system 140 may train a machine learning model to classify compression events as described below with respect to FIG. 8.

The training data may include a dataset that has been featurized and labeled. For example, the dataset may include a plurality of data records, each including information from a different user profile stored in user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning models.

The one or more models are then trained by the training server system 140 using the featurized and labeled training data. In particular, the features of each data record may be used as input into the machine learning models, and the generated output may be compared to labels associated with the corresponding data record. In certain embodiments, the models may compute a loss based on the difference between the generated output and the provided labels. This loss is then used to modify the internal parameters or weights of the model. By iteratively processing each data record for each historical patient, the models may be iteratively refined to generate predictions distinguishing between sensor failure and compression events.

As illustrated in FIG. 1, the training server system 140 deploys these trained models to the analytics engines 114 for use during runtime. For example, the compression event detection module 154 may process signals from the continuous analyte monitoring system and output a determination to determine whether a compression event has occurred and possibly a type of compression event (e.g., shown as output 144 in FIG. 1). In certain embodiments, the output 144 generated by the compression event detection module 154 may be stored in the user profile 118.

FIG. 2 is a diagram 200 conceptually illustrating an example continuous analyte monitoring system 104 including example continuous analyte sensors with sensor electronics, in accordance with certain aspects of the present disclosure. For example, the system 104 may continuously monitor one or more analytes of a user, in accordance with certain aspects of the present disclosure.

The continuous analyte monitoring system 104 includes sensor electronics module 204 and one or more continuous analyte sensors 202 (individually referred to herein as continuous analyte sensor 202 and collectively referred to herein as continuous analyte sensors 202). The sensor electronics module 204 may be in wired or wireless communication (e.g., directly or indirectly) with one or more of display devices 210, 220, 230, and 240. The sensor electronics module 204 may also be in wired or wireless communication (e.g., directly or indirectly) with one or more medical devices, such as medical devices 208 (individually referred to herein as medical device 208 and collectively referred to herein as medical devices 208), or one or more other non-analyte sensors 206 (individually referred to herein as non-analyte sensor 206 and collectively referred to herein as non-analyte sensor 206).

A continuous analyte sensor 202 may include one or more sensors for detecting or measuring analytes. A continuous analyte sensor 202 may be a multi-analyte sensor that continuously measures two or more analytes (e.g., glucose, lactate, potassium, ketone, etc.), or a single analyte sensor that continuously measures a single analyte (e.g., where one continuous analyte sensor 202 is used for measuring glucose and then a second continuous analyte sensor 202 used for measuring lactate, etc.). The continuous analyte sensor 202 may be a non-invasive device, a subcutaneous device, a transcutaneous device, a transdermal device, or an intravascular device. The continuous analyte sensor 202 may continuously measure analyte levels of a user using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The continuous analyte sensor 202 may provided a data stream indicative of the concentration of one or more analytes in the user. The data stream may include raw data signals, which are then converted into a calibrated or filtered data stream, e.g., by the analytics engines 114 as described above, and are used to provide estimated analyte values to the user. The raw data signals may further be analyzed by the compression event detection module 154 to determine whether a compression event is indicated and possibly a type of compression event.

The continuous analyte sensor 202 may be a multi-analyte sensor that continuously measures multiple analytes in a user's body. For example, the continuous multi-analyte sensor 202 may be a single sensor that measures glucose, lactate, ketones, glycerol, potassium (e.g., hyperkalemia), sodium, CO2 or anion-gap, or similar analytes in the user's body.

The sensor electronics module 204 includes electronic circuitry for measuring and processing the continuous analyte sensor data. The sensor electronics module 204 can be physically connected to the continuous analyte sensors 202 and can be integral with (non-releasably attached to) or releasably attachable to the continuous analyte sensors 202. The sensor electronics module 204 may include hardware, firmware, or software that enable measurement of levels of analytes via the continuous analyte sensors 202. For example, the sensor electronics module 204 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, or a processor.

The display devices 210, 220, 230, or 240 may display displayable sensor data, including analyte data, which may be transmitted by the sensor electronics module 204. The sensor electronics module 204 may transmit raw sensor data that is converted to displayable sensor data via one or more of the display devices 210, 220, 230, and 240. The sensor electronics module 204 may convert raw sensor data to displayable sensor data and transmit the displayable sensor data to one or more of the display devices 210, 220, 230, or 240. Each of the display devices 210, 220, 230, or 240 may include a display such as a touchscreen display 212, 222, 232, or 242 for displaying sensor data to a user or for receiving inputs from the user. For example, a graphical user interface (GUI) may be presented to the user for such purposes. The display devices 210, 220, 230, and 240 may include other types of user interfaces such as a voice user interface instead of, or in addition to, a touchscreen display for communicating sensor data to the user of the display device or for receiving user inputs. The display devices 210, 220, 230, and 240 may be examples of display device 107 illustrated in FIG. 1 used to display sensor data to a user of the system of FIG. 1 or to receive input from the user.

The display devices 210, 220, 230, and 240 may display or otherwise communicate the sensor data as it is communicated from the sensor electronics module 204 (e.g., in a customized data package that is transmitted to the display devices 210, 220, 230, and 240 based on their respective preferences), without any additional prospective processing required for calibration and real-time display of the sensor data.

The display devices 210 may include a custom display device specially designed for displaying certain types of displayable sensor data for analyte data received from the sensor electronics module 204. The display device 220 may be a smartphone or a mobile phone using a commercially available operating system (OS) and may display a graphical representation of the continuous sensor data (e.g., including current and historic data). The display device 230 may include a tablet, and the display device 240 may include a smart watch. The medical device 208 may include an insulin delivery device or a blood glucose meter. The display devices 210, 220, 230, and 240 and the medical device 208 may include a desktop or laptop computer (not shown).

Because different display devices provide different user interfaces, content of the data packages (e.g., amount, format, or type of data to be displayed, alarms, and the like) can be customized (e.g., programmed differently by the manufacture or by an end user) for each particular display device. Accordingly, different display devices can be in direct wireless communication with the sensor electronics module 204 (e.g., such as an on-skin sensor electronics module 204 that is physically connected to continuous analyte sensors 202) during a sensor session to enable a plurality of different types or levels of display or functionality for the displayable sensor information.

As mentioned, the sensor electronics module 204 may be in communication with a medical device 208. The medical device 208 may be a passive device. For example, the medical device 208 may be an insulin pump for administering insulin to a user. For a variety of reasons, it may be desirable for such an insulin pump to receive and track analyte values, e.g., glucose values, transmitted from the continuous analyte monitoring systems 104, where the continuous analyte sensor 202 includes at least a glucose sensor.

Further, as mentioned, the sensor electronics module 204 may also be in communication with other non-analyte sensors 206. The non-analyte sensors 206 may include, but are not limited to, a temperature sensor, a force sensor, oxygen sensor, an altimeter sensor, an accelerometer sensor, a gyrometer sensor, a global positioning system (GPS) sensor, a respiratory sensor, electromyogram (EMG) sensor, a galvanic skin response (GSR) sensor, an impedance sensor, an electrocardiogram sensor, a sweat sensor, etc. The non-analyte sensors 206 may also include monitors such as heart rate monitors, blood pressure monitors, pulse oximeters, caloric intake monitors, and medicament delivery devices. One or more of these non-analyte sensors 206 may provide data to the compression event detection module 154 described further below.

The non-analyte sensors 206 may be combined in any other configuration, such as, for example, combined with one or more continuous analyte sensors 202. As an illustrative example, a non-analyte sensor, e.g., a temperature sensor, may be combined with a continuous glucose analyte sensor 202 configured to sense glucose to form a glucose/temperature sensor used to transmit sensor data to the sensor electronics module 204 using common communication circuitry. As another illustrative example, a non-analyte sensor, e.g., a temperature sensor, may be combined with a multi-analyte sensor 202 that measures glucose and, e.g., lactate to form a glucose/lactate/temperature sensor used to transmit sensor data to the sensor electronics module 204 using common communication circuitry.

One or more of the continuous analyte monitoring systems 104, the plurality of display devices, the medical devices 208, or the non-analyte sensors 206 may communicate together wirelessly using one of a variety of wireless communication technologies (e.g., Wi-Fi, Bluetooth, Near Field Communication (NFC), cellular, etc.). A wireless access point (WAP) may be used to couple one or more of the continuous analyte monitoring system 104, the plurality of display devices, the medical devices 208, or the non-analyte sensors 206 to one another. For example, the WAP may provide Wi-Fi, Bluetooth, or cellular connectivity among these devices. NFC may also be used among the devices depicted in the diagram 200 of FIG. 2.

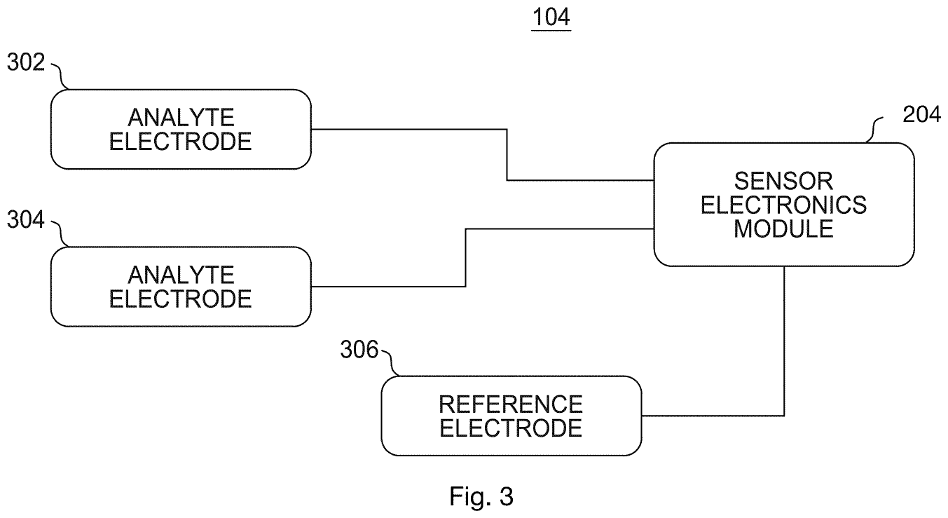

FIG. 3 illustrates an example continuous analyte monitoring systems 104, according to some embodiments disclosed herein. The continuous analyte monitoring system 104 includes the sensor electronics module 204 as well one or more working electrodes that each operate as an analyte sensor. As seen in FIG. 3, the continuous analyte monitoring system 104 includes one or more analyte electrodes 302 and 304 (which may also be referred to as “working electrodes”) and a reference electrode 306. The continuous analyte monitoring system 104 may include any suitable number of analyte electrodes (e.g., one or more analyte electrodes). Each of the electrodes may include a membrane over its surface. The electrodes may be at least partially covered by the same membrane. Alternatively, one or more of the electrodes may not be covered by a membrane as in a bare electrode. The electrodes may be electrically coupled to the sensor electronics module 204 (e.g., the potentiostat and the processor module of the sensor electronics module 204).

The electrodes may be formed of any suitable materials and by any suitable methods. For example, the electrodes may be formed of one or more noble metals, such as platinum, palladium, rhodium, iridium, ruthenium, or platinum/iridium. In some embodiments, the electrodes are carbon-based, and include carbon, carbon/ruthenium, doped diamond, carbon nanotube, graphene, graphite, amorphous carbon, or carbon fiber. In certain embodiments, the electrodes are formed of graphite, gold, conductive polymer, indium tin oxide, or the like. The reference electrode 306 may include silver, silver/silver chloride, or iridium oxide and may be kept currentless. Generally, suitable methods for forming the electrodes include roll-to-roll techniques, screen printing, microfabrication techniques, such as physical vapor deposition, chemical vapor deposition, electrodeposition, lithography, and/or etching techniques. Other methods, including spray deposition or dip-coating, are also contemplated.

The continuous analyte monitoring system 104 may be positioned on a body of a user (e.g., user 102) by inserting some or all of the working electrodes into the body. The working electrodes may be inserted into an adequate insertion site, such as an abdomen or an arm of the user 102, where the working electrodes may be in contact with the blood or interstitial fluid of the user 102.

Different electrodes may detect different analytes. For example, each of the electrodes may include an active surface to facilitate electrochemical sensing of desired analytes. The active surface of each electrode may be voxelated, or partitioned into discrete sections (e.g., cubic sections). Different biorecognition elements (e.g., enzymes, antibodies, aptamers, double-stranded deoxyribonucleic acid (DNA), single-stranded DNA, ribonucleic acid (RNA), oligonucleotides, proteins, cells, microbes, ion-selective materials, etc.), each specific to a different analyte, may be deposited and immobilized on each active surface of each electrode. For example, both glucose oxidase and lactate or uric acid oxidase (and/or other analyte-specific enzymes) may be deposited on the active surface of one electrode. Different enzymes may be immobilized on each voxel of each electrode. In some embodiments, only one type of enzyme is deposited on each voxel of each electrodes. The enzymes may be immobilized via adsorption, entrapment, cross-linking, covalent bonding, or any other suitable immobilization methods.

Each of the electrodes includes enzymes for one specific analyte, while different electrodes may include enzymes for different analytes. In certain embodiments, some of the electrodes include two or more enzymes, such as four or more enzymes, which together enable sensing of a single analyte. For example, where an electrode is configured to sense creatinine, the electrode may include four or more enzymes specific for creatinine. An example of a 1-enzyme sensor electrode may include a lactate-specific electrode, and an example of a 2-enzyme sensor electrode may include a ketone-specific electrode. In the example of FIG. 3, the analyte electrode 302 may be used to measure a level of a first analyte (e.g., glucose). The analyte electrode 304 may be used to measure a level of a second analyte (e.g., lactate). The reference electrode 306 may have a known electrical potential that may serve as a reference potential when measuring or determining the electrical potential of the other electrodes.

During sensing, the deposited enzymes are utilized to convert a respective analyte to an intermediary product (e.g., hydrogen peroxide), which is then oxidized at the surface of the electrodes. The resulting current flow, which is measured by the potentiostat of the sensor electronics module 204 or an ammeter in communication with the potentiostat, is proportional to the analyte concentration. Examples of suitable enzymes include glucose oxidase for sensing glucose species, lactate oxidase for sensing lactate species, lactose oxidase for sensing lactose species, glutamate oxidase for glutamate species, and the like. In addition to enzymes, the active surfaces may further include immobilized redox mediators (e.g., relays) (not shown), which are small electroactive molecules for shuttling electrons between the enzymes and the electrodes. In other embodiments, active surfaces may further include enzyme co-factors, which are compounds used by the enzyme to convert a substrate to a product. In some embodiments, the enzymes are immobilized exclusively over the skive regions of electrodes to minimize or avoid cross-talk between different analytes. In other words, an area of active surface over each of the electrodes may be less than a geometric surface area of the respective working electrode. Maintaining a potential bias at the electrodes may facilitate a near-zero peroxide efflux from the skive region with active consumption of the hydrogen peroxide intermediary.

The continuous analyte monitoring system 104 may use one or more of the signal streams from the analyte electrode 302 and/or the analyte electrode 304 to determine if one of the analyte electrodes 302 or 304 are defective or have detached from the user. In some embodiments, the continuous analyte monitoring system 104 generates a first analyte signal stream using the analyte electrode 302 and a second analyte signal stream using the analyte electrode 304. For example, the analyte electrodes 302 and 304 may generate current flows when particular analytes interact with the surfaces of the analyte electrodes 302 and 304. The potentiostat of the sensor electronics module 204 may measure and report these current flows to the processor of the processor module 214. The processor may then generate the analyte signal streams that represent the measured current flows.

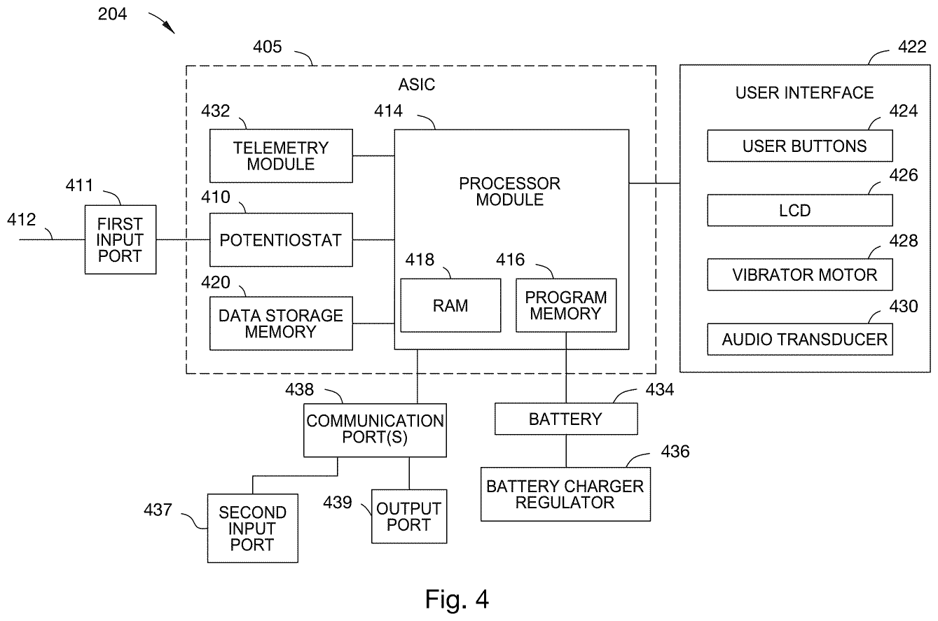

FIG. 4 depicts an example implementation of the sensor electronics module 204, in accordance with some example implementations. The sensor electronics module 204 may include sensor electronics that process sensor information, such as sensor data, and generate transformed sensor data and displayable sensor information, e.g., via a processor module. For example, the processor module may transform sensor data into one or more of the following: filtered sensor data (e.g., one or more filtered analyte concentration values), raw sensor data, calibrated sensor data (e.g., one or more calibrated analyte concentration values), rate of change information, trend information, rate of acceleration/deceleration information, sensor diagnostic information, location information, alarm/alert information, calibration information such as may be determined by factory calibration algorithms as disclosed herein, smoothing or filtering algorithms of sensor data, or the like.

A processor module 414 achieves a substantial portion, if not all, of the data processing, including data processing pertaining to factory calibration. The processor module 414 may include a hardware processor or processor circuitry. The processor module 414 may be integral to the sensor electronics module 204 or may be located remotely, such as in one or more of devices 210, 220, 230, 240 or in a cloud computing platform. The processor module 414 may include smaller subcomponents or submodules. For example, the processor module 414 may include an alert module (not shown) or prediction module (not shown), or any other suitable module that may be utilized to efficiently process data. When the processor module 414 includes submodules, the submodules may be located within the processor module 414, including within the sensor electronics module 204 or other associated devices (e.g., 210, 220, 230, 240). For example, the processor module 414 may be located at least partially within a cloud-based analyte processor or elsewhere in a network.

The processor module 414 may calibrate the sensor data, and the data storage memory 420 may store the calibrated sensor data points as transformed sensor data. Moreover, the processor module 414 may wirelessly receive calibration information from a display device, such as devices 210, 220, 230, 240, to enable calibration of the sensor data from the sensor 12. Furthermore, the processor module 414 may perform additional algorithmic processing on the sensor data (e.g., calibrated or filtered data or other sensor information), and the data storage memory 420 may store the transformed sensor data or sensor diagnostic information of the algorithms. The processor module 414 may store and use calibration information determined from a factory calibration, as described below.

The sensor electronics module 204 may include an application-specific integrated circuit (ASIC) 405 coupled to a user interface 422. The ASIC 405 may further include a potentiostat 410, a telemetry module 432 for transmitting data from the sensor electronics module 204 to one or more devices, such as devices 210, 220, 230, 240, or other components for signal processing and data storage (e.g., processor module 414 and data storage memory 420). Although FIG. 4 depicts ASIC 405, other types of circuitry may be used as well, including field programmable gate arrays (FPGA), one or more microprocessors that provide some (if not all of) the processing performed by the sensor electronics module 204, analog circuitry, digital circuitry, or a combination thereof.

In the example of FIG. 4, through a first input port 411 for sensor data the potentiostat 410 is coupled to a continuous analyte sensor 10, such as a glucose sensor to generate sensor data from the analyte. The potentiostat 410 may also provide via data line 412 a voltage to the continuous analyte sensor 10 to bias the sensor for measurement of a value (e.g., a current and the like) indicative of the analyte concentration in a host (also referred to as the analog portion of the sensor). The potentiostat 410 may have one or more channels depending on the number of working electrodes at the continuous analyte sensor 10.

The potentiostat 410 may include a resistor that translates a current value from the sensor 10 into a voltage value, or a current-to-frequency converter (not shown) may also integrate continuously a measured current value from the sensor 10 using, for example, a charge-counting device. An analog-to-digital converter (not shown) may digitize the analog signal from the sensor 10 into so-called “counts” to allow processing by the processor module 414. The resulting counts may be directly related to the current measured by the potentiostat 410, which may be directly related to an analyte level, such as a glucose level, in the host.

The telemetry module 432 may be operably connected to the processor module 414 and may provide the hardware, firmware, or software that enable wireless communication between the sensor electronics module 204 and one or more other devices, such as display devices, processors, network access devices, and the like. A variety of wireless radio technologies that can be implemented in the telemetry module 432 include Bluetooth, Bluetooth Low-Energy, ANT, ANT+, ZigBee, IEEE 802.11, IEEE 802.16, cellular radio access technologies, radio frequency (RF), infrared (IR), paging network communication, magnetic induction, satellite data communication, spread spectrum communication, frequency hopping communication, near field communications, or the like. The telemetry module 432 may include a Bluetooth chip, although Bluetooth technology may also be implemented in a combination of the telemetry module 432 and the processor module 414.

The processor module 414 may control the processing performed by the sensor electronics module 204. For example, the processor module 414 may process data (e.g., counts), from the sensor, filter the data, calibrate the data, perform fail-safe checking, or the like.

The processor module 414 may include a digital filter, such as for example an infinite impulse response (IIR) or a finite impulse response (FIR) filter. This digital filter may smooth a raw data stream received from the sensor 10. Generally, the digital filters are programmed to filter data sampled at a predetermined time interval (also referred to as a sample rate). When the potentiostat 410 measures the analyte (e.g., glucose or the like) at discrete time intervals, these time intervals determine the sampling rate of the digital filter. The potentiostat 410 may measure continuously the analyte, for example, using a current-to-frequency converter. In these current-to-frequency converter implementations, the processor module 414 may be programmed to request, at predetermined time intervals (acquisition time), digital values from the integrator of the current-to-frequency converter. These digital values obtained by the processor module 414 from the integrator may be averaged over the acquisition time due to the continuity of the current measurement. As such, the acquisition time may be determined by the sampling rate of the digital filter.

The processor module 414 may further include a data generator (not shown) that generates data packages for transmission to devices, such as the display devices 210, 220, 230, 240. Furthermore, the processor module 414 may generate data packets for transmission to these outside sources via the telemetry module 432. In some example implementations, the data packages may, as noted, be customizable for each display device, or may include any available data, such as a time stamp, displayable sensor information, transformed sensor data, an identifier code for the sensor or sensor electronics module 204, raw data, filtered data, calibrated data, rate of change information, trend information, error detection or correction, or the like.

The processor module 414 may also include a program memory 416 and other memory 418. The processor module 414 may be coupled to a communications interface, such as a communication port 438, and a source of power, such as a battery 434. Moreover, the battery 434 may be further coupled to a battery charger or regulator 436 to provide power to sensor electronics module 204 or charge the battery 434.

The program memory 416 may be implemented as a semi-static memory for storing data, such as an identifier for a coupled sensor 10 (e.g., a sensor identifier (ID)) and for storing code (also referred to as program code) to configure the ASIC 405 to perform one or more of the operations/functions described herein. For example, the program code may configure processor module 414 to process data streams or counts, filter, perform the calibration methods described below, perform fail-safe checking, and the like.

The memory 418 may also be used to store information. For example, the processor module 414 including memory 418 may be used as the system's cache memory, where temporary storage is provided for recent sensor data received from the sensor. In some example implementations, the memory may include memory storage components, such as read-only memory (ROM), random-access memory (RAM), dynamic-RAM, static-RAM, non-static RAM, easily erasable programmable read only memory (EEPROM), rewritable ROMs, flash memory, and the like.

The data storage memory 420 may be coupled to the processor module 414 and may store a variety of sensor information. In some example implementations, the data storage memory 420 stores one or more days of continuous analyte sensor data. For example, the data storage memory may store 1, 2, 3, 4, 5, 6, 7, 8, 9, 10, 11, 12, 13, 14, 15, or 30 days (or more days) of continuous analyte sensor data received from the sensor 10. The stored sensor information may include one or more of the following: a time stamp, raw sensor data (one or more raw analyte concentration values), calibrated data, filtered data, transformed sensor data, or any other displayable sensor information, calibration information (e.g., reference BG values or prior calibration information such as from factory calibration), sensor diagnostic information, and the like.

The user interface 422 may include a variety of interfaces, such as one or more buttons 424, a liquid crystal display (LCD) 426, a vibrator 428, an audio transducer (e.g., speaker) 430, a backlight (not shown), or the like. The components that include the user interface 422 may provide controls to interact with the user (e.g., the host). One or more buttons 424 may allow, for example, toggle, menu selection, option selection, status selection, yes/no response to on-screen questions, a “turn off” function (e.g., for an alarm), an “acknowledged” function (e.g., for an alarm), a reset, or the like. The LCD 426 may provide the user with, for example, visual data output. The audio transducer 430 (e.g., speaker) may provide audible signals in response to triggering of certain alerts, such as present or predicted hyperglycemic and hypoglycemic conditions. Audible signals may be differentiated by tone, volume, duty cycle, pattern, duration, or the like. The audible signal may be silenced (e.g., acknowledged or turned off) by pressing one or more buttons 424 on the sensor electronics module 204 or by signaling the sensor electronics module 204 using a button or selection on a display device (e.g., key fob, cell phone, or the like).

Although audio and vibratory alarms are described with respect to FIG. 4, other alarming mechanisms may be used as well. For example, in some example implementations, a tactile alarm is provided including a poking mechanism that “pokes” or physically contacts the patient in response to one or more alarm conditions.

The battery 434 may be operatively connected to the processor module 414 (and possibly other components of the sensor electronics module 204) and provide the necessary power for the sensor electronics module 204. The battery may be a lithium manganese dioxide battery, however any appropriately sized and powered battery can be used (e.g., AAA, nickel-cadmium, zinc-carbon, alkaline, lithium, nickel-metal hydride, lithium-ion, zinc-air, zinc-mercury oxide, silver-zinc, or hermetically-sealed). The battery may be rechargeable. Multiple batteries can be used to power the system. In yet other implementations, the receiver can be transcutaneously powered via an inductive coupling, for example.

A battery charger or regulator 436 may receive energy from an internal or external charger. The battery regulator (or balancer) 436 regulates the recharging process by bleeding off excess charge current to allow all cells or batteries in the sensor electronics module 204 to be fully charged without overcharging other cells or batteries. The battery 434 (or batteries) may be charged via an inductive or wireless charging pad, although any other charging or power mechanism may be used as well.

One or more communication ports 438, also referred to as external connectors, may be provided to allow communication with other devices, for example a PC communication (com) port can be provided to enable communication with systems that are separate from, or integral with, the sensor electronics module 204. The communication port, for example, may include a serial (e.g., universal serial bus or “USB”) communication port, and allow for communicating with another computer system (e.g., PC, personal digital assistant or “PDA,” server, or the like). The sensor electronics module 204 may transmit historical data to a PC or other computing device (e.g., an analyte processor as disclosed herein) for retrospective analysis by a patient or physician. As another example of data transmission, factory information may also be sent to the algorithm from the sensor or from a cloud data source.

The one or more communication ports 438 may further include a second input port 437 in which calibration data may be received, and an output port 439 which may be employed to transmit calibrated data, or data to be calibrated, to a receiver or mobile device. FIG. 4 illustrates these aspects schematically. It will be understood that the ports may be separated physically, but in alternative implementations a single communication port may provide the functions of both the second input port and the output port.

In some continuous analyte sensor systems, an on-skin portion of the sensor electronics may be simplified to minimize complexity or size of on-skin electronics, for example, providing only raw, calibrated, or filtered data to a display device configured to run calibration and other algorithms required for displaying the sensor data. However, the sensor electronics module 204 (e.g., via processor module 414) may be implemented to execute prospective algorithms used to generate transformed sensor data or displayable sensor information, including, for example, algorithms that: evaluate a clinical acceptability of reference or sensor data, evaluate calibration data for best calibration based on inclusion criteria, evaluate a quality of the calibration, compare estimated analyte values with time corresponding measured analyte values, analyze a variation of estimated analyte values, evaluate a stability of the sensor or sensor data, detect signal artifacts (noise), replace signal artifacts, determine a rate of change or trend of the sensor data, perform dynamic and intelligent analyte value estimation, perform diagnostics on the sensor or sensor data, set modes of operation, evaluate the data for aberrancies, or the like.

Although separate data storage and program memories are shown in FIG. 4, a variety of configurations may be used as well. For example, one or more memories may be used to provide storage space to support data processing and storage requirements at sensor electronics module 204.

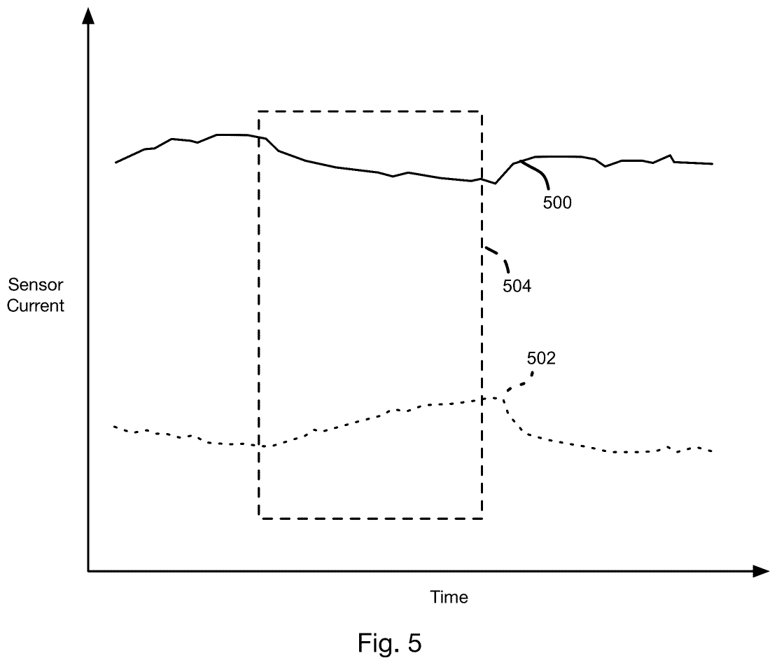

FIG. 5 illustrates a plot 500 of the output of a glucose sensor and plot 502 of the output of a lactate sensor such as may be one of the continuous analyte sensors 202 (e.g., along with a glucose sensor) or incorporated into a multi-analyte sensor 202 (e.g., a multi-analyte sensor able to measure both glucose and lactate levels). As shown, during a compression event 504, the output of the glucose sensor will fall while the output of the lactate sensor will rise. This inverse movement may result from a lack of oxygen triggering anaerobic respiration or some other physiological process. In the absence of a compression event 504, the output of the glucose sensor and output of the lactate sensor will move in correlation over the long term, with some lag.

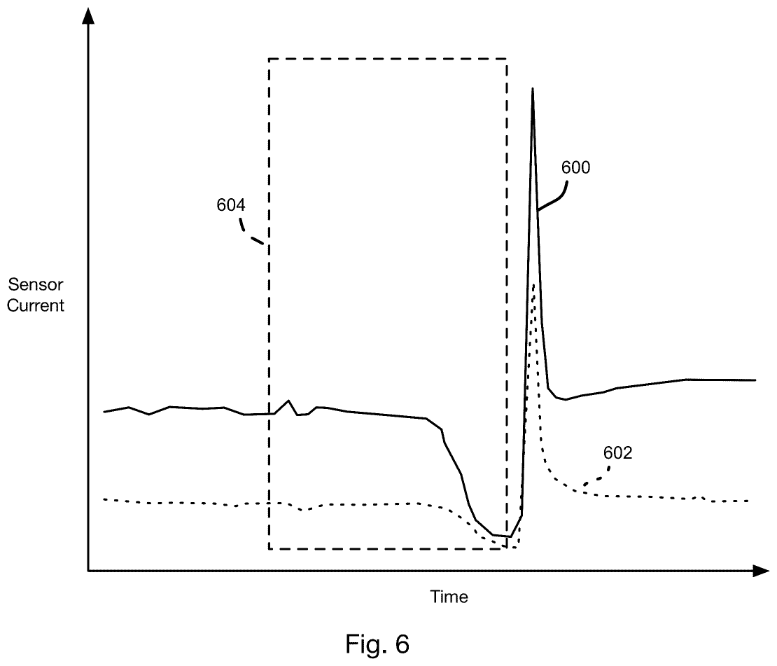

FIG. 6 illustrates a plot 600 of the output of a glucose sensor and plot 602 of the output of a lactate sensor such as may be incorporated into the continuous analyte sensor 202. During an acute compression event 604, the outputs of the glucose sensor and lactate sensor will vary drastically and in correlation. As is apparent, the outputs of the glucose sensor and lactate sensor have a “biphasic” response to an acute compression event: a steep drop during the compression event and a sharp spike following the compression event.

As is apparent from FIGS. 5 and 6, the outputs of the glucose sensor and lactate sensor during and immediately after a compression event 504, 604 can exhibit clear patterns of inverse correlation or a correlated biphasic fluctuation. These patterns may be used, possibly with other information, to compensate for compression events by either (a) adjusting sampled values or (b) suppressing use of sampled values (“blanking”). Compensating for compression events is an example of an ameliorating action that may be taken in response to compression events. Compensation as described below may additionally or alternative include other ameliorating actions such as generating an alert, marking samples corresponding to a compression event as having low confidence or performing some other action to account for the possibility of inaccuracy due to the compression event.

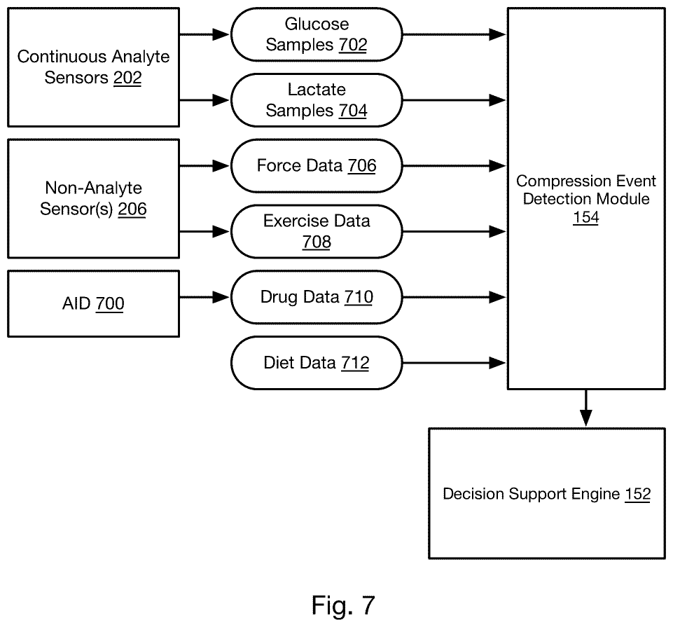

For example, referring to FIG. 7, the compression event detection module 154 may receive inputs from one or more continuous analyte sensors 202, one or more non-analyte sensors 206, and possibly from an automatic insulin device (AID) 700, such as an insulin pump supplying subcutaneous insulin to the user of the continuous analyte monitoring system 104.

For example, the compression event detection module 154 may receive glucose samples 702 and lactate samples 704 from the continuous analyte sensor 202. The glucose samples 702 and lactate samples 704 may be samples of the current output by analyte electrodes 302, 304 whether in raw form or following one or more pre-processing steps such as amplification or filtering.

The compression event detection module 154 may receive a force data 706 output from the non-analyte sensors 206, such as from a force sensor implemented as a load cell, pressure-sensitive switch, force-sensitive resistor, or any other device capable of sensing force exerted thereon. The force sensor may be, for example, mounted in or on a housing of the continuous analyte monitoring system 104 such that force exerted on the force sensor correlates to force exerted on the analyte electrodes 302, 304 and the tissue surrounding the analyte electrodes 302, 304.

The compression event detection module 154 may receive exercise data 708 from the non-analyte sensors 206. For example, the non-analyte sensors 206 may include an accelerometer and the exercise data 708 may be the output of the accelerometer or a representation of the output of the accelerometer, such as a step count, estimated distance walked, run, or swum. The non-analyte sensors 206 may include a heart rate monitor and the exercise data 706 may be the output of the heart rate monitor or a representation of the output of the accelerometer, such as average heart rate for a time period, maximum heart rate for a time period, amount of time spent above a threshold heart rate or in a heart rate range, an estimate of calories burned based on measurements of heart rate, or other data derived from the output of the heart rate monitor. The exercise data 708 may include data facilitating interpretation of the output of an accelerometer or heart rate monitor, such as weight, biological gender, resting heart rate, or other data.

The AID 700, the user, or some other individual or device may supply drug data 710 to the compression event detection module 154. The drug data 710 may include an amount of insulin supplied subcutaneously to the user. The amount may be represented as a volume injected and a time of injection. The amount may be represented as a rate of insulin delivery by the AID 208 during a time period, such as an hour, 15 minutes, 5 minutes, or some other interval.

The compression event detection module 154 may receive diet data 712. The diet data 712 may include data describing calories, protein, carbohydrates, sodium or other macro- and micronutrients by the user. The diet data 712 may be input by the user or other individual into an application on a display device 107, 210, 220, 230, 240 or some other device.

The compression event detection module 154 receives some or all of the above-referenced data and provides an output to the decision support engine 152. The output may include raw data from the analyte electrodes 302, 304 without modification, or with modification compensating for a compression event. The output may include adjusted data obtained by adjusting the raw data to compensate for a compression event. The output may be modified to adjust data corresponding to a compression event or include blanked data for the compression event. Blanked data may include data that indicates that no glucose and/or lactate measurement are available for a time period, such as for a sampling period or block of sampling periods. For example, the sampling period may be every 30 seconds or some other time interval.

The decision support engine 152 will then process the output as described above in order to provide alerts regarding hyper- or hypo-glycemic events and/or providing recommendations with respect to nutrition and administration of insulin.

Referring to FIG. 8, the compression event detection module 154 may include a compression classifier 800. The compression classifier 800 may receive some or all of the data described above as being input to the compression event detection module 154. In some embodiments, the compression classifier 800 receives only the glucose samples 702 and lactate samples 704. In other embodiments, the compression classifier 800 receives only the glucose samples 702 and lactate samples 704 along with force data 706.

The compression classifier 800 may be a machine learning model such as a logistic regression machine learning model, decision tree machine learning model, Bayesian machine learning model, neural network, deep neural network, convolution neural network, or any other type of machine learning model.

The compression classifier 800 may be trained with training data entries. Each training data entry may include, as a desired output, a classification, e.g., a first value indicating no compression event, a second value indicating a compression event that is compensatable, and a third value indicating an acute compression event that should result in blanking. As used herein “compensatable” may be understood as meaning that glucose samples can be adjusted to compensate for a compression event, such as using an assumed inverse correspondence with lactate samples. Each training data entry may include, as inputs, some or all of the glucose samples 702, lactate samples 704, force data 706, exercise data 708, drug data 710, and diet data 712 measured for a patient for a time period that either does not include a compression event or for a time period including a compression event, whether acute or not. The inputs may include data for just the time period during which the compression event occurred or may include data for a time period extending after the compression event and possibly before and after the compression event.

The data input to the compression classifier 800 may include data derived from some or all of the data input to the compression event detection module 154. For example, for data represented as a series of samples or measurement, the data input to the compression classifier 800 may be a smoothed version of such data, such as using an exponential smoothing function or other type of smoothing function. The data input to the compression classifier 800 may include features extracted from the data, such as mean, variability, outlier count (e.g., Z-score<−2), maximum value, minimum value, 25^{th} percentile, 75^{th} percentile, standard deviation, or other statistical characterization.

The compression classifier 800 may be trained using the training data entries. For example, the inputs of each training data entry may be processed using the compression classifier 800 to obtain an output that is compared to the desired output of the training data entry. A training algorithm may then update parameters of the compression classifier 800 based on the comparison. The compression classifier 800 is therefore trained to output a classification 802 for a given set of inputs indicating whether the inputs correspond to a compensatable compression event, an acute compression event, or the absence of a compression event.

The classification 802 and other data, such as the glucose samples 702 and lactate samples 704 may be input to compensation logic 804. The compensation logic may do nothing where the classification 802 indicates no compression event. The compensation logic may output blanked data or suppress output of the glucose samples 702 and lactate samples 704 where the classification 802 indicates an acute compression event. The samples blanked may include all samples within the time window that were processed by the compression classifier 800 to obtain the classification. The samples blanked may include all samples that are below a threshold, such as a threshold that corresponds to samples that would otherwise be interpreted as a hypoglycemic event when converted to glucose estimates. For example, where the samples are measurements of current from an analyte electrode 302 measuring glucose, the threshold may be less than 600 pico Amperes (pA), less than 500 pA, or less than 400 pA. For example, the threshold may be less than or equal to 325.52 pA.

The compensation logic 804 may adjust the glucose samples 702 and or lactate samples 704 when the classification 802 indicates a compensatable compression event. Compensation may include applying a function to both the glucose samples 702 and lactate samples 704 to obtain adjusted values for the glucose samples. For example, the function may increase a glucose sample in correspondence with a magnitude of a mean glucose value and the value of the glucose sample and a magnitude of the corresponding lactate sample (e.g., same sample period) and a mean lactate value. The mean glucose value and mean lactate value may be for a time window preceding the time window in which the compression event is detected. In other embodiments, the glucose sample may be adjusted by substituting the mean glucose value or obtaining a weighted average of the glucose sample and the mean glucose value. Adjusting the glucose sample may include processing the glucose samples using a smoothing filter, such as a Kalman filter.

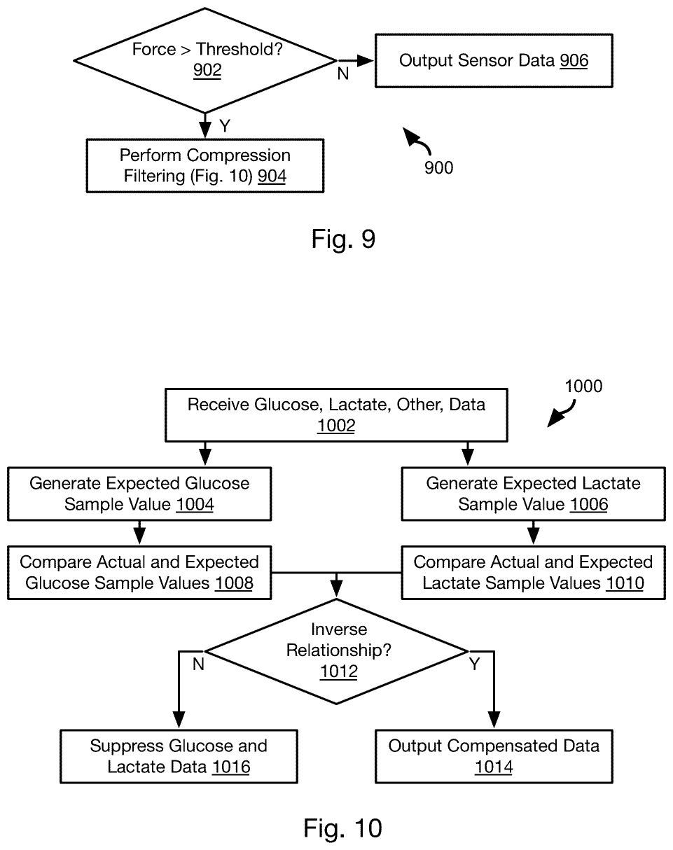

FIGS. 9 and 10 illustrate methods that may be used in place of or along with the compression classifier 800. Referring specifically to FIG. 9, in embodiments where the non-analyte sensors 206 include a force sensor, the method 900 may be used to make an initial assessment as to whether a compression event may have occurred. If the output of the force sensor is found, at step 902, to exceed a threshold, then compression filtering is performed at step 904. Compression filtering may include performing filtering such that glucose measurements that are artificially low due to compression events are either adjusted or blanked. An example approach for performing compression filtering is described below with respect to FIG. 10.

If the force is not found to be greater than the threshold, the method 900 may include outputting, at step 906, sensor data from the continuous analyte sensors 202 to the decision support engine 152 without performing compression filtering.

FIG. 10 illustrates a method 1000 for performing compression filtering. The method 1000 may be invoked according to the method 900 or based on some other criteria. For example, the method 1000 may be performed in all cases with the compression filtering being performed such that, in the absence of a compression event, the effect of the compression filtering will be insignificant or function only to smooth the samples output from the continuous analyte sensors. A criteria for invoking the method 1000 may include the current output of the electrode 302 detecting glucose falling below 300 pA, less than 200 pA, or less than 100 pA, such as below 90 pA for a single sample, all samples within a time window (e.g., 5, 10, or 60 minutes) or a threshold percentage of samples within the time window (e.g., at least 60 percent, at least 80 percent, or at least 90 percent).

The method 1000 includes receiving, at step 1002, a stream of data. The stream of data may include various types of data include glucose samples 702 and lactate samples 704. The glucose samples 702 and lactate samples 704 may be raw data (e.g., current measurements from analyte electrodes 302, 304) or processed data (e.g., estimates of glucose and lactose concentration). In some embodiments, other data is included in the stream of data such as some or all of force data 706, exercise data 708, drug data 710, and diet data 712.

Step 1002 may include receiving the samples for glucose and lactate for a current sample period (n), which are referred to herein as Ĝ[n] and \hat{L}[n], respectively. Other types of data such as force data 706 (F), exercise data 708 (E), diet data 712 (D), and drug data 710 (Dr) may also be time varying but at a slower rate such that such data does not vary for multiple sample periods. Accordingly, step 1002 may include receiving updated values for these types of data or using the same data from a previous iteration of the method 1000.

The method 1000 includes generating, at step 1004, an expected glucose sample value and generating, at step 1006, an expected lactate sample value. For example, steps 1004 and 1006 may include evaluating equations (1) and (2) to calculate predicted first derivatives for glucose and lactate (Ġ[n] and \dot{L}[n], respectively) for the current sample period (n). Expected values for the current samples of glucose and lactate (G[n] and L[n]) may then be calculated according to equations (3) and (4) based at least in part on previous values for glucose and lactate (Ĝ[n−1] and \hat{L}[n−1]). The values of Ġ[n−1] and \dot{L}[n−1] used in equations (3) and (4) may be values calculated according to (1) and (2) in a previous iteration or calculated based on actual values: Ġ[n−1]=(Ĝ[n−1]−Ĝ[n−2])/Δt and \dot{L}[n−1]=(\hat{L}[n−1]−\hat{L}[n−2])/Δt, where Δt is the duration of the sampling period for the glucose and lactate samples, such as a value between 1 minute and 20 seconds, such as 30 seconds. The coefficients A, B, C, M, N, O, P, Q, R, S, T and U may be determined using previously received data for the user of the continuous analyte monitoring system 104 or for a different user or group of users. The coefficients may be calculated using a curve fitting algorithm, linear regression, or other technique.

$$\dot{G} [ n ] = A * F + B * \dot{L} [ n - 1 ] + C * \hat{G} [ n - 1 ] + M * E + N * D + O * Dr \qquad ( 1 )$$

$$\dot{L} [ n ] = P * F + Q * \dot{G} [ n - 1 ] + R * \hat{L} [ n - 1 ] + S * E + T * D + U * Dr \qquad ( 2 )$$

$$G [ n ] = \hat{G} [ n - 1 ] + \dot{G} [ n - 1 ] * Δ t \qquad ( 3 )$$

$$L [ n ] = \hat{L} [ n - 1 ] + \dot{L} [ n - 1 ] * Δ t \qquad ( 4 )$$

The method 1000 may include comparing the actual and expected sample values for glucose at step 1008 and comparing the actual and expected sample values for lactate at step 1010. In particular, steps 1008 and 1010 may include calculating difference values, such as ΔG=G[n]−Ĝ[n] and ΔL=L[n]−\hat{L}[n], respectively.

The method 1000 may include evaluating, at step 1012, whether the ΔG and ΔL exhibit an inverse relationship, i.e., opposite signs. Step 1012 may include evaluating whether ΔG and ΔL exhibit a specific inverse relationship: falling glucose (negative ΔG) and rising lactate (positive ΔL) corresponding to a compression event as shown in FIG. 5. Step 1012 may require that the inverse relationship or the specific inverse relationship be true for multiple sample periods n or for a percentage (e.g., at least 50, at least 70, or at least 90) of the sample periods within a time window, such as a 5, 10, 20, 30 or 60 minute window.

If the inverse relationship is found, the method 1000 may include outputting, at step 1014, compensated data. Compensated data may include outputting the expected values for glucose and/or lactate G[n] and L[n]) rather than the actual sample values Ĝ[n] and \hat{L}[n]. Compensation may include outputting an average or weighted average of the expected values for glucose and/or lactate G[n] and L[n]) and the actual sample values Ĝ[n] and \hat{L}[n].

The method 1000 is an example implementation of a compression filter. Other approaches may also be used. For example, a Kalman filter may be used to calculate expected values and also be used to compensate for compressions using the inherent smoothing provided by a Kalman filter. Other filtering or smoothing approaches may also be used to calculate the expected values for glucose and lactate.

If the inverse relationship is not found at step 1012, the method 1000 may include suppressing, at step 1016, output of the actual glucose and possibly lactate samples. In particular, where an above-threshold force is detected according to the method 900 or based on some other criteria and the inverse relationship is not found, an acute compression event may have occurred (see FIG. 6) such that compensation is not helpful or possible. As noted above, suppressing may include outputting nothing or blanking a value for a sample period to indicate that the sample for the sample period has been suppressed.

In some embodiments, the method 1000 may be modified to omit the use of lactate sample values. For example, terms relating to lactate may be removed from equation (1). Step 1012 may be modified to omit evaluation of an inverse relationship between glucose and lactate. For example, step 1012 may be modified to include evaluating a magnitude of the difference ΔG between the actual and expected glucose sample values. For example, if ΔG is less than a difference threshold, step 1016 will be executed, otherwise step 1014 may be performed.

In another embodiment, a rate of change of the actual glucose sample value Ĝ[n] may be evaluated with respect to a rate of change threshold. For example, if a negative rate of change having a magnitude greater than the rate of change threshold, step 1016 will be performed, otherwise step 1014 may be performed.

In yet another embodiment, a difference between the actual glucose sample Ĝ[n] may be compared to a reference value, such as a moving average of actual glucose samples for a preceding time window, such as the last 5 minutes, 10 minutes, 20 minutes, one hour, or other time interval. If the actual glucose sample value Ĝ[n] is below the reference value by more than a reference threshold, step 1016 will be performed, otherwise step 1014 may be performed.

The methods disclosed herein include one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

The term “continuous,” as used herein, is a broad term, and is used in its ordinary sense, and can mean continuous, semi-continuous, continual, periodic, intermittent, regular, etc.

The terms “continuous analyte sensor,” “continuous multi-analyte sensor,” “continuous glucose sensor,” and “continuous lactate sensor,” as used herein, are broad terms, and are used in their ordinary sense, and refer without limitation to a device that continuously measures a concentration of an analyte or calibrates the device (e.g., by continuously adjusting or determining the sensor's sensitivity and background), for example, at time intervals ranging from fractions of a second up to, e.g., 1, 2, or 5 minutes, or longer.

The terms “sensitivity” or “sensor sensitivity,” as used herein, are broad terms, and are used in their ordinary sense, and refer without limitation to an amount of signal produced by a certain concentration of a measured analyte, or a measured species (e.g., H₂O₂) associated with a measured analyte (e.g., glucose or lactate). For example, a sensor may have a sensitivity of from about 1 to about 300 pico Amperes (pA) of current for every 1 mg/dL of glucose analyte.

The term “sensor data,” as used herein, is a broad term, and is used in its ordinary sense, and refers without limitation to any data associated with a sensor, such as a continuous analyte or continuous multi-analyte sensor. Sensor data includes a raw data stream, or simply data stream, of analog or digital signal directly related to a measured analyte from an analyte sensor (or other signal received from another sensor), as well as calibrated or filtered raw data. The terms “sensor data point” and “data point” refer generally to a digital representation of sensor data at a particular time. The terms broadly encompass a plurality of time spaced data points from a sensor, such as a continuous analyte sensor, which includes individual measurements taken at time intervals ranging from fractions of a second up to, e.g., 1, 2, or 5 minutes or longer. In another example, the sensor data includes an integrated digital value representative of one or more data points averaged over a time period. Sensor data may include calibrated data, smoothed data, filtered data, transformed data, or any other data associated with a sensor.

The term “sensor electronics,” as used herein, is a broad term, and is used in its ordinary sense, and refers without limitation to components, e.g., hardware or software, of a device configured to process sensor data.

Although certain embodiments herein are described with reference to management of diabetes, diabetes management is only an example of one application for which the present systems and methods may be utilized. The systems and methods described herein can also be used for managing one or more other diseases or conditions, which may or may not include diabetes. For example, the systems and methods described herein can be utilized for managing kidney disease, liver disease, and other types of diseases or conditions.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “including as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. An apparatus comprising:
one or more sensors configured to sense a first analyte and a second analyte within tissue of a user; and
a controller operably coupled to the one or more sensors, the controller configured to:
evaluate first samples representing measurements of the first analyte using the one or more sensors and second samples representing measurements of the second analyte using the one or more sensors with respect to one another to determine whether the first samples and the second samples indicate compression of the tissue; and
if the first samples and the second samples indicate compression of the tissue, perform an ameliorating action.

2. The apparatus of claim 1, wherein the controller is configured to perform the ameliorating action by compensating for the compression of the tissue with respect to the first samples.

3. The apparatus of claim 1, wherein the ameliorating action includes blanking the first samples.

4. The apparatus of claim 1, wherein the first analyte is glucose and the second analyte is lactate.

5. The apparatus of claim 1, further comprising a force sensor, the controller further configured to:
evaluate an output of the force sensor with respect to a threshold condition; and
evaluate the first samples and the second samples to determine whether the first samples and the second samples indicate the compression of the tissue in response to the output of the force sensor meeting the threshold condition.

6. The apparatus of claim 1, wherein the controller is further configured to evaluate the first samples and the second samples to determine whether the first samples and the second samples indicate the compression of the tissue by evaluating whether the first samples and the second samples have an inverse correlation.

7. The apparatus of claim 6, wherein the controller is further configured to adjust values of the first samples if the first samples and the second samples have the inverse correlation.

8. The apparatus of claim 6, wherein the controller is further configured to:
calculate a first expected value for a current first sample of the first samples;
calculate a second expected value for a current second sample of the second samples;
calculate a first difference between the first expected value and the current first sample;
calculate a second difference between the second expected value and the current second sample; and
determine that the first samples and the second samples have the inverse correlation in response to the first difference and the second difference having opposite signs.

9. The apparatus of claim 8, wherein the first expected value is a function of one or more samples of the first samples preceding the current first sample and one or more samples of the second samples preceding the current second sample.

10. The apparatus of claim 9, further comprising a force sensor;
wherein the first expected value is a function of an output of the force sensor.

11. The apparatus of claim 9, wherein the first expected value is a function of diet data for the user.

12. The apparatus of claim 9, wherein the first expected value is a function of drug data for the user.

13. The apparatus of claim 12, wherein the drug data is an amount of subcutaneous insulin delivered to the user.

14. The apparatus of claim 9, wherein the first expected value is a function of exercise data for the user.

15. The apparatus of claim 9, wherein the controller is further configured to compensate for the compression of the tissue with respect to the first samples based on the first expected value.

16. The apparatus of claim 6, wherein the controller is further configured to blank the first samples in response to the first samples and the second samples not having the inverse correlation.

17. The apparatus of claim 1, wherein the controller is further configured to evaluate the first samples and the second samples to determine whether the first samples and the second samples indicate the compression of the tissue by evaluating the first samples and the second samples by processing the first samples and the second samples using a machine learning model.

18. The apparatus of claim 17, wherein the controller is configured to evaluate the first samples and the second samples to determine whether the first samples and the second samples indicate the compression of the tissue by processing, using the machine learning model, at least one of force data, exercise data, drug data, and diet data.

19. A non-transitory computer-readable medium storing executable code that, when executed by one or more processing devices, causes the one or more processing devices to:
receive first samples from a first analyte sensor positioned within tissue of a user;
receive second samples from a second analyte sensor positioned within the tissue of the user;
evaluate the first samples and the second samples with respect to one another to determine whether the first samples and the second samples indicate compression of the tissue; and
if the first samples and the second samples indicate compression of the tissue, compensate for the compression of the tissue with respect to the first samples.

20. The non-transitory computer-readable medium of claim 19, wherein the executable code, when executed by the one or more processing devices, further causes the one or more processing devices to compensate for the compression of the tissue by at least one of adjusting values of the first samples or blanking the first samples.
